(19) United States
(12) Patent Application Publication
(10) Pub. No.: US 2013/0060165 A1
(43) Pub. Date: Mar. 7, 2013

(54) Device for Detecting The Curvature of the Skin Surface of a Human or an Animal Subject

(71) Applicant: Ka Wing Chan, Hong Kong (CN)

(72) Inventor: Ka Wing Chan, Hong Kong (CN)

(21) Appl. No.: 13/569,199

(22) Filed: Aug. 8, 2012

Related U.S. Application Data
(60) Provisional application No. 61/530,391, filed on Sep. 1, 2011.

(51) Int. Cl. A61B 5/20; A61B 5/103

(52) U.S. Cl. 600/587

(57) ABSTRACT

This invention relates to a device for detecting the curvature change of the skin surface of a human or an animal subject, comprising a sensing unit and a processing unit. The device can be used for monitoring distention level of the bladder of the subject and so parents may find it useful for toilet training their children by knowing the status of bladders of their children from the device. It is also suitable for other people such as patients suffering from uncontrollable urination, physically disabled patients and/or comatose patients, and even pet owners.

### CROSS-REFERENCE TO RELATED APPLICATION

This application claims the benefit of U.S. Provisional Application No. 61/530,391 filed 1 Sep. 2011, which is hereby incorporated by reference herein in its entirety.

### FIELD OF INVENTION

This invention relates to a device for detecting the curvature change of the skin surface of a human or an animal subject, in particular for monitoring distention level of the bladder of the subject.

### BACKGROUND OF INVENTION

Parents want to train their children to go to toilet independently. Such act can increase the self-confidence of the children and it is also the entry requirement for nursery schools. However, most of the current toilet training systems are passive and inefficient. For instance, one of such systems trains children to stay for a certain time on their own potties while they can watch TV and read books on the potties simultaneously. Unfortunately, children usually do not want to go to toilet because they do not have enough urine or they have already discharged urine before the training and so toilet training fails. Also, children usually fails to show clear signal of urination to the parents until the very last moment and so parents cannot run the training as expected effectively; thus they may need an indicator for indicating the fullness of the children's bladders.

Devices for measuring the volume of urine in bladders are available. For example, U.S. Pat. No. 5,058,591 and U.S. Pat. No. 4,926,871 disclose techniques for the measurement of the volume of a human bladder utilizing ultrasound transducer. However, the implementation of ultrasonic transducer in such device may make it expensive and too bulky to be readily installed on children or ones in need thereof, and may also interrupt their movement.

### SUMMARY OF INVENTION

In light of the foregoing background, it is an object of the present invention to provide a device to facilitate the monitoring of distention level of the bladder.

Accordingly, the present invention, in one aspect, is a device for detecting the curvature change of the skin surface of a human or an animal subject, comprising:

a sensing unit adapted flexibly attachable onto a predetermined area of said skin surface for detecting said curvature change of said predetermined area and generating data for said curvature change; and

a processing unit connected to said sensing unit and adapted to receive said data and trigger the generation of an indication signal when said data is beyond a pre-defined threshold value.

In an exemplary embodiment of the present invention, said processing unit of said device further comprises:

an amplifying subunit connected to said sensing unit and adapted to amplify the data generated by said sensing unit;

a filtering subunit connected to said amplifying subunit and adapted to filter said amplified data;

a converting subunit connected to said filtering subunit and adapted to convert said filtered data into a form analyzable by said analyzing unit;

an analyzing subunit connected to said converting subunit and adapted to compare said converted data from said converting subunit with said pre-defined threshold value and generate a working signal to an indicating subunit when said converted data is beyond said pre-defined threshold value; and

an indicating subunit connected to said analyzing subunit and adapted to generate said indication signal on receiving said working signal from said analyzing subunit.

In an exemplary embodiment of the present invention, said device further comprises a housing wherein said sensing unit and said processing unit are disposed in said housing; said housing comprises a film attachable to said skin surface and soft enough to allow the detection of said curvature change by said sensing unit. In yet a further exemplary embodiment, said housing is made of water-proof material and/or biocompatible material.

In another exemplary embodiment of the present invention, said indication signal is a visual signal, an audio signal, or a combination thereof.

In another exemplary embodiment of the present invention, said indication signal is transmitted via a wireless transmission to an external device. In a further exemplary embodiment, said external device is a computer, a mobile device, or a combination thereof.

In another exemplary embodiment of the present invention, said device further comprises a fastening unit for flexibly attaching said device to said skin surface.

Another aspect of the present invention provides a method for detecting the curvature change of the skin surface of a human or an animal subject comprising the steps of:

providing the device for detecting the curvature change of the skin surface of a human or an animal subject as described above;

attaching a sensing unit of the device onto a predetermined area of the detected object for detecting said curvature change of said predetermined area;

obtaining data from said sensing unit representative of said curvature change;

transmitting said data to a processing unit; and

triggering said processing unit to generate an indication signal when said data is beyond a pre-defined threshold value.

In an exemplary embodiment of the present invention, the triggering step of the processing unit further comprises the steps of:

amplifying said data from said sensing unit by an amplifying subunit;

filtering said amplified data by a filtering subunit;

converting said filtered data by a converting subunit to a form analyzable by an analyzing subunit;

comparing said converted data from said converting subunit with said pre-defined threshold value and generating a working signal when said converted data is beyond said pre-defined threshold value by said analyzing subunit;

transmitting said working signal to an indicating subunit; and

sending said indication signal to a user.

In another exemplary embodiment of the present invention, said indication signal is a visual signal, an audio signal, or any combination thereof.

In yet another exemplary embodiment of the present invention, said indication signal is a wireless signal receivable by an external device.

A further aspect of the present invention provides a method for toilet training of a subject in need thereof comprising the steps of:

providing the device for detecting the curvature change of the skin surface of a human or an animal subject as described above;

attaching said sensing unit of the device to the lower abdominal region of said subject for detecting the curvature change of said lower abdominal region and generating data representative of the change;

triggering said processing unit to generate an indication signal when said data is beyond a pre-defined threshold value; and

alerting a user on receiving said indication signal to take corresponding action.

In an exemplary embodiment of the present invention, said indication signal is a visual signal, an audio signal, or any combination thereof.

There are many advantages to the present invention. For instance, in a toilet training program, upon attaching the device described in the present invention to the children, parents will easily be alerted by the device when the bladders of their children are full, so that they can take or teach their children to toilet immediately. As such, children will learn how to do independent toileting quickly with more confidence.

Besides, the small size of the device allows the user to move more freely without much hindrance. It is also inexpensive and easy to operate for any users.

There are some other advantages of the present invention. For example, children usually suffer from bedwetting and this would be a demerit when they grow up. On using the device descried in the present invention, parents can readily be noticed on the distention level of the bladder of their children even when they are sleeping, and can thus take their children to toilet when necessary to avoid bedwetting.

Apart from children, the device of the present invention is also suitable for other people such as patients suffering from uncontrollable urination, physically disabled patients, and/or comatose patients. On using the device of the present invention, the use of diaper can be reduced to minimal and so they can live more comfortably with dignity.

Pet owners can also find the device useful in toilet training their pets on how to use their pet potties to avoid dirtying their houses and public areas.

### BRIEF DESCRIPTION OF THE DRAWINGS

FIG. 1 is a schematic illustration of a device according to one embodiment of the present invention.

FIG. 2 is a block diagram showing the major components and the connection thereof for a device according to the same embodiment of the present invention.

FIGS. 3a and 3b illustrate the device in use when it is attached to the pre-defined location of the subject in need thereof according to the same embodiment of the present invention.

FIGS. 4a and 4b illustrate flow diagrams of the main operation steps of the device according to two embodiments of the present invention.

### DETAILED DESCRIPTION OF THE INVENTION

As used herein and in the claims, “comprising” means including the following elements but not excluding others.

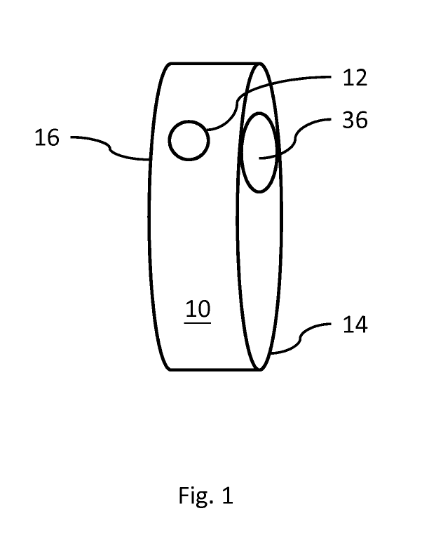

Referring first to FIG. 1, the first embodiment of the present invention is a device 10 for detecting the curvature change of the skin surface of a human or an animal subject comprising a housing 14 in which a sensing unit 30 and a processing unit 31 (not shown in FIG. 1) are disposed therein.

In an exemplary embodiment of the present invention, the housing 14 comprises a film 16. The outside surface of the film 16 is attached to a pre-determined area of the subject when the device 10 is in use, while on the inside surface of the film 16, the sensing unit 30 is disposed thereon. In one exemplary embodiment, the pre-determined area is the area of the lower abdominal region of a human above the pubic bone and in close proximity to the bladder. The film 16 is made of material soft enough to allow the sensing unit 30 to effectively detect the curvature change of the pre-determined area.

In a further exemplary embodiment, the housing 14 is made of water-proof material and/or biocompatible material to protect the device 10 from potential contamination by urine as such contamination may cause current leakage of the device.

In another exemplary embodiment of the present invention, the device 10 further comprises a fastening unit for flexibly attaching the device to the pre-determined area. In a further exemplary embodiment, the fastening unit is made of an adhesive and biocompatible material and disposed on the outside of the film 16.

In an exemplary embodiment of the present invention as illustrated in FIG. 1, an indicating subunit 36 is disposed on the housing 14 for generating a visual signal (in which case the indicating subunit 36 may be a display or an LED light), an audio signal (in which case the indicating subunit 36 may be a speaker), or any combination thereof.

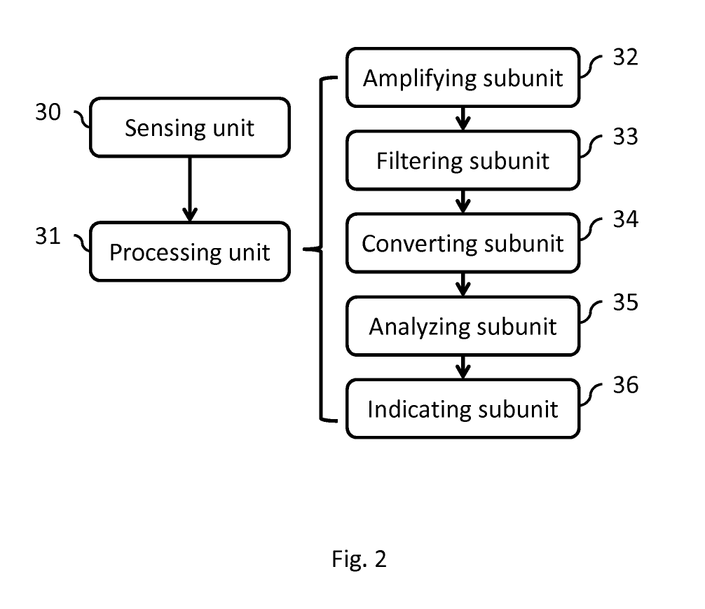

Referring now to FIG. 2, the sensing unit 30 is a sensor for detecting the curvature change of the pre-determined area and generating data for the curvature change. In another exemplary embodiment, the generated data for the curvature change is an analog signal.

The processing unit 31 is connected to the sensing unit 30 for receiving the data generated from the sensing unit 30. It triggers the generation of an indication signal when the data is beyond a pre-defined threshold value.

In a further exemplary embodiment, the processing unit 31 further comprises an amplifying subunit 32, a filtering subunit 33, a converting subunit 34, an analyzing subunit 35 and an indicating subunit 36. In one exemplary embodiment as illustrated in FIG. 2, the filtering subunit 33 is connected to the amplifying subunit 32; the converting subunit 34 is connected to the filtering subunit 33; the analyzing subunit 35 is connected to the converting subunit 34; and the indicating subunit 36 is connected to the analyzing subunit 35.

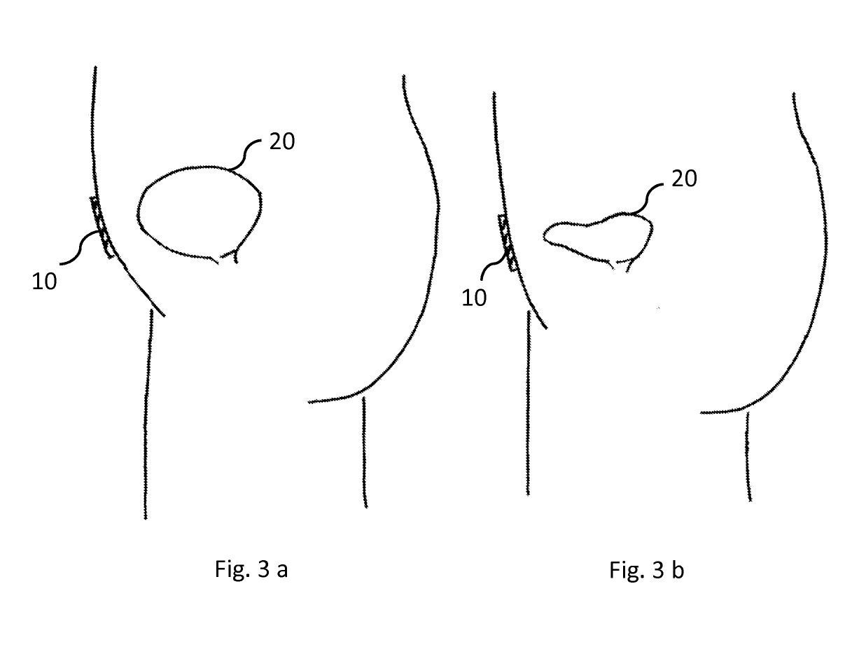

During operation, the device 10 is attached to the pre-determined area for detecting the curvature change of that area. In the exemplary embodiment as shown in FIGS. 3a & 3b, the device 10 is placed in the lower abdominal region of the human subject. The bladder is in the state of fullness or near fullness in FIG. 3a and in the state of emptiness or near emptiness in FIG. 3b.

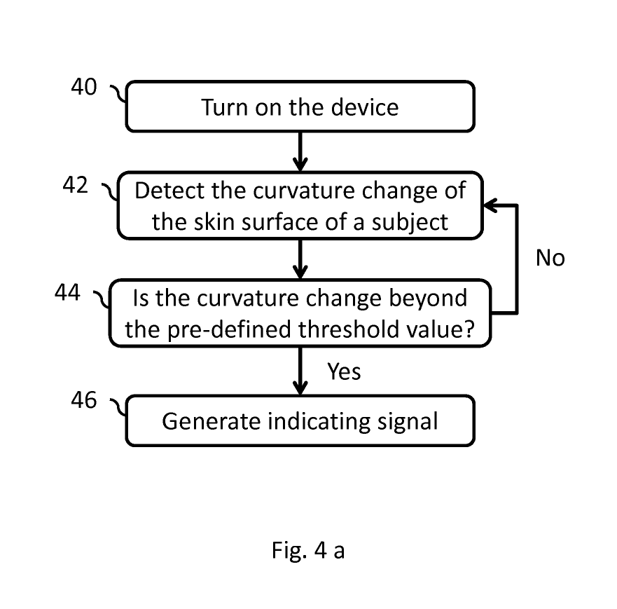

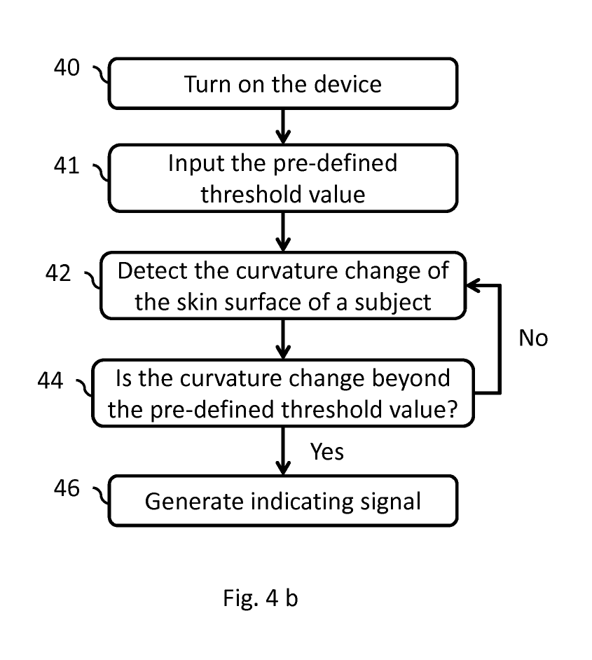

In an exemplary embodiment, the flow of operation of the device 10 is shown in FIGS. 4a & 4b. Upon switching on the device 10 (box 40), the sensing unit 30 begins to detect the curvature change of the pre-determined area (box 42) and a data representative of the curvature change is generated by the sensing unit 30. The data is then transmitted to the processing unit 31 and checked whether it is beyond the pre-defined threshold value (box 44). In the scenario that the data exceeds the pre-defined threshold value, the processing unit 30 is triggered to generate the indication signal (box 46).

In a further exemplary embodiment, upon being transmitted to the processing unit 31, the data is amplified by the amplifying subunit 32 and filtered by the filtering subunit 33. The filtered data is converted by the converting subunit 34 into a form analyzable by the analyzing subunit 35 and is compared with the pre-defined threshold value. A working signal is generated by the analyzing subunit 35 and transmitted to the indicating subunit 36 when the converted data is beyond the pre-defined threshold value. The indicating subunit 36 then generates the indication signal on receiving the working signal from the analyzing subunit 35. The aforesaid data flow inside the processing unit 31 is shown in FIG. 2 for ease of illustration.

In an exemplary embodiment of the present invention as illustrated in FIG. 1, a switch 12 is disposed on the housing 14 for switching on/off and/or resetting the device 10.

In an exemplary embodiment, the pre-defined threshold value can be pre-set in the device 10 or can be inputted by the user (box 41) as shown in FIG. 4b. In another exemplary embodiment, the pre-defined threshold value is determined based on the clinical data from a large number and variety of subjects, such as children, physical disabled patients and pets. In another exemplary embodiment, the pre-defined threshold value is tailor-determined for a particular subject based on the physiological data of that subject.

In yet another exemplary embodiment, in defining the threshold value, the state of an empty bladder is first recorded, followed by calculating the threshold value based on a mapping table. In one exemplary embodiment, the mapping table for different types of subjects is shown in Table 1. The threshold may be set as 6 for a child, 4.7 for an adult, or 11.4 for a pet. In another exemplary embodiment, the pet used in the construction of the mapping table (Table 1) is a dog.

TABLE 1
A mapping table for calculating the threshold value

| Degree of | | Sensor Output | Threshold (Voltage) | | |
|---|---|---|---|---|---|
| Curvature | | (Voltage) | Child | Adult | Pet |
| (Flat) | 1 | 0.01 | 5.0 | 4.0 | 10.0 |
| | 2 | 0.02 | 5.0 | 4.0 | 10.2 |
| | 3 | 0.03 | 5.0 | 4.2 | 10.4 |
| | 4 | 0.04 | 5.5 | 4.2 | 10.6 |
| | 5 | 0.05 | 5.5 | 4.5 | 10.8 |
| | 6 | 0.06 | 5.5 | 4.5 | 11.0 |
| | 7 | 0.07 | 5.5 | 4.7 | 11.2 |
| | 8 | 0.08 | 6.0 | 4.7 | 11.4 |
| | 9 | 0.09 | 6.0 | 4.9 | 11.6 |
| (Bended) | 10 | 0.1 | 6.0 | 4.9 | 11.8 |

In another exemplary embodiment of the present invention, the indication signal is transmitted via a wireless transmission (e.g. radio frequency transmission) to an external device such as a computer, a mobile device with a wireless receptor or any combination thereof. In view of the aforesaid description, the application of the device of the instant invention can be extended to the aspect of toilet training for children and pets. For instance, the device 10 can be attached to the lower abdominal region, in close proximity of the bladder, of a child or a pet for detecting the curvature change of that region. When the curvature change is beyond a pre-defined threshold value, referring to the state in which the bladder is full or nearly full, an indication signal is generated to alert the parents or pet owners to take corresponding training actions, for example, taking the child to toilet or the pet to the pet potty.

The preferred embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the external device to receive the transmitted indication signal may be designed to be located in the vicinity of the subject or person taking care of the subject, which may be a wrist-mounted device with a wireless receptor worn by the subject or the caretaker. The wrist-mounted device can generate an alert to the subject or the caretaker when the device detects that the curvature change is beyond the threshold value.

Also, it may be clear to one skilled in the art that a specific operation method to switch on/off the device to avoid it being mistakenly switched on/off can be designed. For instance, a double-press or a long-press on the switch can be designed as the operating signal to the device.

What is claimed is:

1. A device for detecting the curvature change of the skin surface of a human or an animal subject, comprising:
a. a sensing unit adapted flexibly attachable onto a predetermined area of said skin surface for detecting said curvature change of said predetermined area and generating data for said curvature change; and
b. a processing unit connected to said sensing unit and adapted to receive said data and trigger the generation of an indication signal when said data is beyond a pre-defined threshold value.

2. The device according to claim 1, wherein said processing unit comprises:
a. an amplifying subunit connected to said sensing unit and adapted to amplify said data generated by said sensing unit;
b. a filtering subunit connected to said amplifying subunit and adapted to filter said amplified data;
c. a converting subunit connected to said filtering subunit and adapted to convert said filtered data into a form analyzable by said analyzing subunit;
d. an analyzing subunit connected to said converting subunit and adapted to compare said converted data from said converting subunit with said pre-defined threshold value and generate a working signal to an indicating subunit when said converted data is beyond said pre-defined threshold value; and
e. an indicating subunit connected to said analyzing subunit and adapted to generate said indication signal on receiving said working signal from said analyzing subunit.

3. The device according to claim 1 further comprises a housing wherein said sensing unit and said processing unit are disposed therein; said housing comprises a film attachable to said skin surface and soft enough to allow the detection of said curvature change by said sensing unit.

4. The device according to claim 3, wherein said housing is made of water-proof material and/or biocompatible material.

5. The device according to claim 1, wherein said indication signal is selected from a group consisting of a visual signal, an audio signal and a combination thereof.

6. The device according to claim 1, wherein said indication signal is transmitted via wireless transmission to an external device.

7. The device according to claim 6, wherein said external device is selected from a group consisting of a computer, a mobile device, and any combination thereof.

8. The device according to claim 1, wherein said device further comprises is a fastening unit for flexibly attaching said device to said skin surface.

9. A method for detecting the curvature change of the skin surface of a human or an animal subject comprising the steps of:
i) providing a device according to claim 1;
ii) attaching a sensing unit of said device onto a predetermined area of the detected object for detecting said curvature change of said predetermined area;
iii) obtaining data from said sensing unit representative of said curvature change;
iv) transmitting said data to a processing unit; and
v) triggering said processing unit to generate an indication signal when said data is beyond a pre-defined threshold value.

10. The method according to claim 9, wherein said step (v) further comprises the steps of:
vi) amplifying said data from said sensing unit by an amplifying subunit;
vii) filtering said amplified data by a filtering subunit;
viii) converting said filtered data by a converting subunit to a form analyzable by an analyzing subunit;
ix) comparing said converted data from said converting subunit with said pre-defined threshold value and generating a working signal when said converted data is beyond said pre-defined threshold value by said analyzing subunit;
x) transmitting said working signal to a indicating subunit; and
xi) sending said indication signal to a user.

11. The method according to claim 9, wherein said indication signal is selected from a group consisting of a visual signal, an audio signal, and any combination thereof.

12. The method according to claim 9, wherein said indication signal is a wireless signal receivable by an external device.

13. A method for toilet training of a subject in need thereof comprising the steps of:
i) providing a device according to claim 1;
ii) attaching said sensing unit to the lower abdominal region of said subject for detecting the curvature change of said lower abdominal region and generating data representative of the change;
iii) triggering said processing unit to generate an indication signal when said data is beyond a pre-defined threshold value; and
iv) alerting a user on receiving said indication signal to take corresponding action.

14. The method according to claim 13, wherein said pre-defined threshold value is tailor-determined for a particular subject based on the physiological data of said particular subject.

15. The method according to claim 13, wherein said indication signal is selected from a group consisting of a visual signal, an audio signal, and any combination thereof.
